(19) United States
(12) Patent Application Publication
(10) Pub. No.: US 2010/0167399 A1
(43) Pub. Date: Jul. 1, 2010

(54) METHOD OF GENERATING REGULATORY T CELLS USING CORD BLOOD AND ADULT BLOOD CD14+ MONOCYTE CELLS

(75) Inventor: Makio Iwashima, River Forest, IL (US)

Correspondence Address: HARTMAN & HARTMAN, P.C., 552 EAST 700 NORTH, VALPARAISO, IN, 46383, US

(73) Assignee: LOYOLA UNIVERSITY CHICAGO, Maywood, IL (US)

(21) Appl. No.: 12/644,140

(22) Filed: Dec. 22, 2009

Related U.S. Application Data
(60) Provisional application No. 61/141,032, filed on Dec. 29, 2008.

(51) Int. Cl. C12N 5/0783

(52) U.S. Cl. 435/372.3

(57) ABSTRACT

A method of inducing stable and functional Tregs from human umbilical cord blood and adult blood without requiring the expansion of pre-existing Tregs. The method utilizes CD14+ monocyte cells present in or isolated from cord blood or adult blood to induce functional Tregs from T cells, and particularly T cells that express CD4+, which may also be obtained from cord blood or adult blood. The developed Tregs are long-lasting and maintain their suppressive functions.

### CROSS REFERENCE TO RELATED APPLICATIONS

This application claims the benefit of U.S. Provisional Application No. 61/141,032, filed Dec. 29, 2008, the contents of which are incorporated herein by reference.

### BACKGROUND OF THE INVENTION

The present invention generally relates to methods for producing regulatory T cells (Tregs), and more particularly to a method for producing Tregs from human cord blood and adult blood. The Tregs produced by the method of this invention are generally stable in vitro and functional, and believed to provide a new procedure for treatment of autoimmune disorders, organ transplantation, and allergic disease.

Tregs are a specialized subpopulation of T cells whose function is to suppress the immune responses of other cells (leukocytes) in the immune system in order to tolerate self-antigens and prevent autoimmune disease. As such, methods of producing Tregs are desired for the treatment of autoimmune disorders, organ transplantation, and allergic disease. Existing methods for effectively producing long-lasting Tregs are generally achieved through the expansion of pre-existing Tregs. Such methods require purification of Tregs by flow cytometery using surface markers, entailing a relatively cumbersome and expensive cell purification process. Another disadvantage with Tregs induced by existing methods (for example, transforming growth factor beta, or TGF-β) is that they are not stable and do not maintain the expression of Foxp3, an essential protein required for Treg survival and/or function.

It would be desirable if a simpler process existed for inducing human Tregs that did not require purification or the use of flow cytometer, yet was capable of generating Tregs that are long lasting and maintain their suppressive functions.

### BRIEF DESCRIPTION OF THE INVENTION

The present invention provides a method capable of inducing stable and functional Tregs from human umbilical cord blood and adult blood without requiring the expansion of pre-existing Tregs, such as through the purification of pre-existing Tregs by flow cytometery.

According to the invention, CD14+ monocyte cells present in or isolated from cord blood or adult blood are used to induce functional Tregs from T cells, including those that express CD4+ and CD8+, which may also be obtained from cord blood or adult blood. A technical effect of the invention is the capability of a relatively simple process to induce human Tregs without the use of cumbersome and expensive cell purification processes. Data from investigations leading to this invention evidenced that the developed Tregs are long lasting, keep their phenotype for more than six weeks, and maintain their suppressive functions. If cord blood is used, the method does not require any cell fractionation.

Other aspects and advantages of this invention will be better appreciated from the following detailed description.

### BRIEF DESCRIPTION OF THE DRAWINGS

FIG. 1 plots cell subsets present in cord blood and adult blood characterized by surface antigen expression.

FIG. 2 plots the results of inducing Foxp3+ regulatory T cells (Tregs) with cord blood CD14+ monocyte cells using a method of the invention.

FIG. 3 compares the induction of Foxp3+ Tregs by cord blood (“CB only”), cord blood CD14+ monocyte cells (“CB CD14”), and adult CD14+ monocyte cells (“Adult CD14”) achieved with methods within the scope of the invention.

FIG. 4 plots the results of inducing Foxp3+ Tregs with cord blood cells by a non-direct interaction technique.

FIG. 5 plots the results of inducing Foxp3+ CD8 Tregs from cord blood.

FIG. 6 plots the suppression activity of CD8+ Tregs derived from cord blood.

FIG. 7a is a graph evidencing the inhibition of Treg induction in the presence of a TGF-β receptor inhibitor during Foxp3+ Treg induction with CD14+ monocyte cells.

FIG. 7b is a graph evidencing the presence of activin C in a culture supernatant of cord blood stimulated with anti-CD3 to induce Tregs, and the absence of activin C in a culture supernatant of cord blood T cells stimulated by anti-CD3 and anti-CD28 antibody-coated polystyrene beads.

### DETAILED DESCRIPTION OF THE INVENTION

In investigations leading to the present invention, cord blood and adult blood cells were separated into lymphocyte/monocyte fraction by centrifugation using Ficoll®. CD14+ monocyte cells were then purified by magnetic sorting using antibody-coated magnetic beads, and the T cell lymphocyte and CD14+ monocyte cell fractions were combined in a culture to which anti-CD3 antibody and recombinant human IL-2 cytokine were added. Culture medium containing IL-2 but not anti-CD3 was changed approximately every three to four days. The cells were cultured for about one to two weeks, after which functional regulatory CD4+Foxp3+ T cells (Tregs) were isolated from the culture.

Based on observations from this and subsequent investigations, it was concluded that, in place of the whole lymphocyte/monocyte fraction used in the investigation, purified CD4+ naive T cells and CD14+ cells could have been used as a source of Tregs that express CD4+Foxp3+. Furthermore, it was predicted the anti-CD3 antibody, which is known to stimulate all T cells, could have been replaced by or supplemented with other suitable stimulators, such as other antigenic proteins, allogenic MHC (major histocompatibility complex) molecules, and/or antigen peptides (both self and non-self).

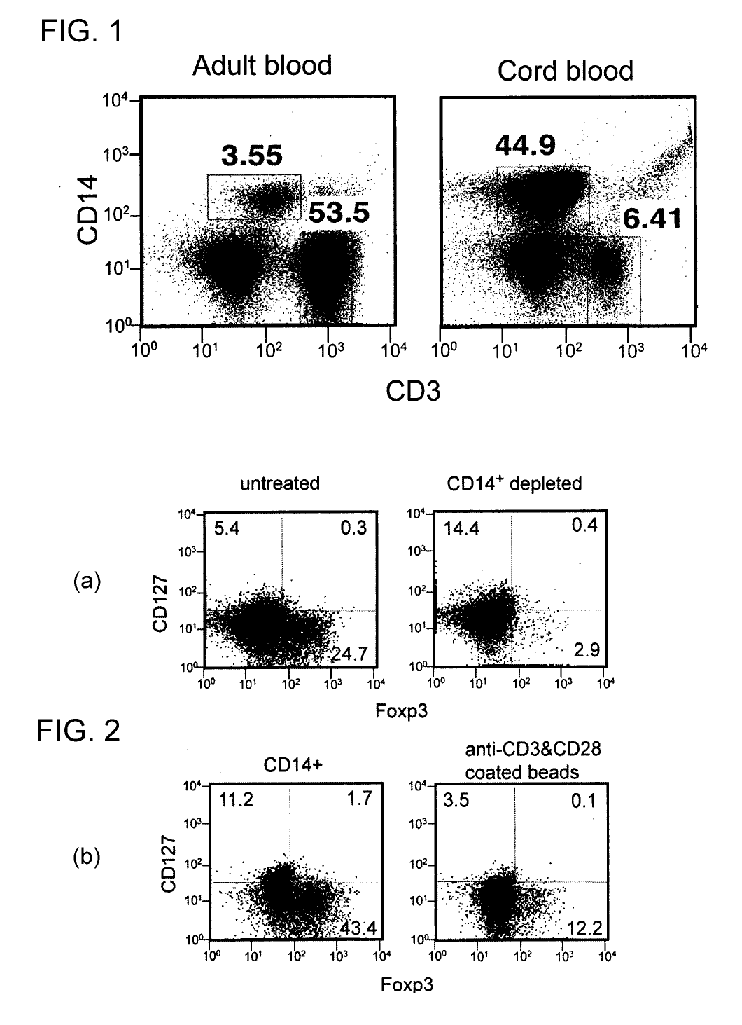

In a further investigation, CD14+ monocyte cells were shown to be required for induction of Foxp3+(CD4+) regulatory T cells, referred to as naturally-occurring regulatory T cells (nTregs). The investigation was carried out to further analyze the cells responsible for induction of Tregs from cord blood. In the investigation, cell subsets present in cord blood and adult blood were characterized by surface antigen expression (FIG. 1). Cord blood and adult peripheral blood cells were treated to remove red cells, then stained with antibodies against CD3 (x-axis) and CD14 (y-axis) followed by flow cytometery. The numbers in FIG. 1 indicate the frequency of CD3+ T cells and CD14+ monocytes in each sample. The data in FIG. 1 show that cord blood contains a significantly higher level of CD14+ monocyte fractions than adult peripheral blood (about 45% compared to less that 4% CD14+ monocyte cells). Because of this difference, it was hypothesized that CD14+ monocyte cells may be required in nTreg induction by cord blood cells, and experiments were carried out to test the role of CD14+ monocyte cells in the induction of nTregs.

In one investigation, CD4+ T cells from cord blood were mixed with a specimen of irradiated total cord blood cells and with a specimen of cord blood cells depleted of CD14+ monocyte cells. The total cord blood specimen and CD14+ depleted blood specimen were then treated with anti-CD3 antibody and IL-2 for Treg induction. After two weeks of culture, cells were analyzed from each specimen for Foxp3+ expression (FIG. 2a). While treatment of total cord blood cells (“untreated”) with anti-CD3 antibody and IL-2 led to induction of cells that expressed Foxp3+ (about 25% of total cells), the removal of CD14+ monocyte cells (“CD14+ depleted”) significantly reduced the level of cells that expressed Foxp3+ (about 3%). The data in FIG. 2a clearly demonstrated that the presence of CD14+ cells in cord blood is required for induction of Foxp3+ regulatory T cells.

Another investigation was then conducted to determine whether CD14+ cord blood cells are sufficient to induce Tregs. For this purpose, CD14+ monocyte cells were again isolated from cord blood and mixed with CD4+ T cells from cord blood. As a control, plastic beads coated with anti-CD3 plus anti-CD28 antibodies were also used to stimulate a specimen of CD4+ T cells. Stimulation of the CD4+ T cells with the CD14+ cord blood cells resulted in about 45% of the CD4+ T cells becoming Foxp3+ Tregs (left panel of FIG. 2b). On the other hand, only 12% of the same T cell population expressed Foxp3+ when stimulated with anti-CD3 and anti-CD28 coated plastic beads (right panel of FIG. 2b). Thus, the data strongly suggested that CD14+ monocyte cells are alone sufficient to induce Foxp3+ Tregs from CD4+ T cells.

Successful induction of nTregs by cord blood CD14+ monocyte cells prompted another investigation to examine the potential for Treg induction using adult peripheral blood CD14+ monocyte cells. Since the major difference between cord blood and adult peripheral blood is the frequency of CD14+ monocyte cells, it was hypothesized that the low frequency of CD14+ monocyte cells in adult blood may reduce Treg induction from adult blood. In a sample of cord blood, the level of CD14+ monocyte cells was as high as 50% of total nucleated cells, whereas the level of CD14+ monocyte cells was less than 5% in a sample of adult blood. A hypothesis was developed that enriched CD14+ monocyte cells from adult blood, when mixed with CD4+ T cells, would induce Foxp3+ Tregs as was observed with cord blood CD14+ monocyte cells. To test this hypothesis, CD14+ monocyte cells were isolated from adult peripheral blood as well as from cord blood. These isolated cells were mixed with CD4+CD25− T cells from another adult peripheral blood sample. Because these peripheral T cells will respond to allogenic antigen presented by CD14+ monocyte cells, anti-CD3 antibody was not added to the culture. Induction of Foxp3+ Tregs with this system would indicate that these Tregs will be effective regulatory T cells against allogenic tissue graft. As a control for CD14+ monocyte cells, peripheral T cells were stimulated with anti-CD3 and anti-CD28 coated polystyrene beads.

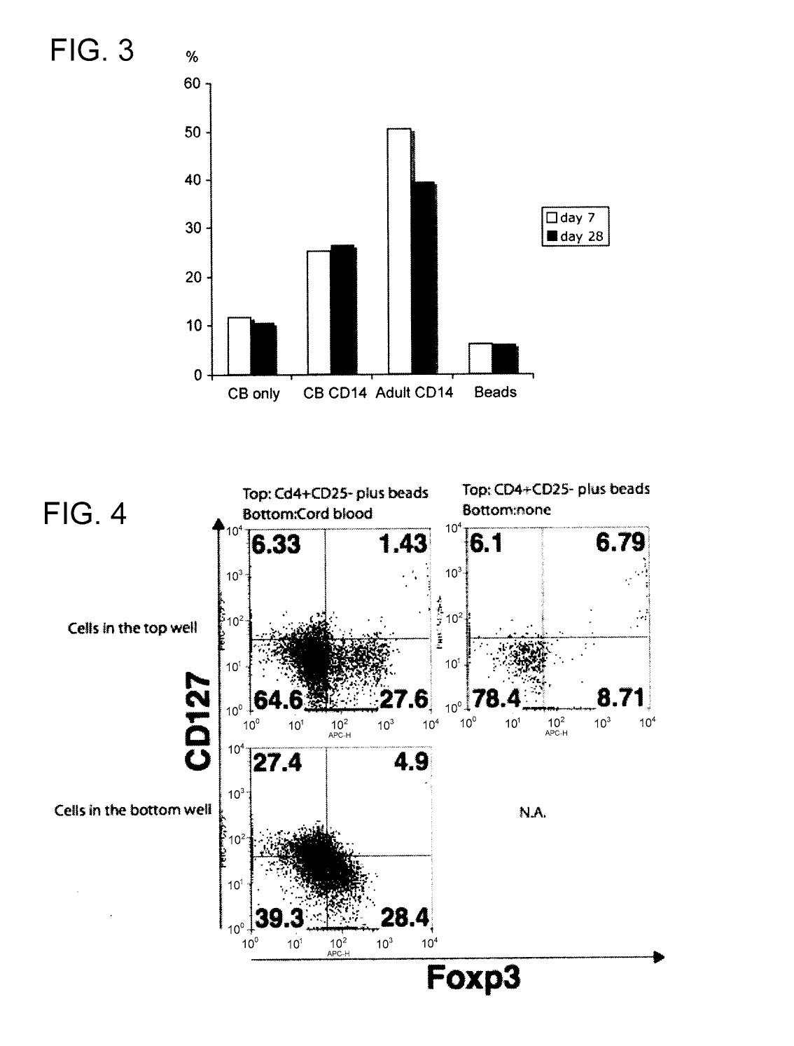

As indicated in FIG. 3, under these conditions cord blood-derived CD14+ monocyte cells (“CB CD14”) induced about 25% to about 33% of Foxp3+ Tregs from the adult blood sample after seven days. Surprisingly, in the mixture of allogenic CD14+ monocyte cells from adult blood and CD4+CD25− T cells (“Adult CD14”), approximately 50% of the T cells became Foxp3+ Tregs during the same period. These numbers were significantly higher than what were observed in samples stimulated with anti-CD3 and anti-CD28 coated beads (about 5% Foxp3+ Tregs). Since the observed Foxp3+ Tregs might reflect a transient expression of Foxp3+ by activated T cells, the culture was maintained for three additional weeks without extra stimulation. In samples that had been stimulated with adult CD14+ monocyte cells (“Adult CD14”), the level of Foxp3+ Tregs remained high (about 35%) and were comparable to the “CB only” and “CB CD14” samples treated with cord blood CD14+ monocyte cells (about 12 to about 24%). On the other hand, the cells stimulated with the anti-CD3 and anti-CD28 coated polystyrene beads (“Beads”) remained low in Foxp3+ expression and contained only about 5% of Foxp3+ Tregs. The data represented in FIG. 3 evidenced that adult CD14+ monocyte cells, when enriched, are as potent as cord blood CD14+ monocyte cells in induction of Tregs in vitro.

The data presented above demonstrated that both adult and cord blood CD14+ monocyte cells are capable of inducing Foxp3+ Tregs from adult peripheral T cells, and particularly T cells that express CD4+. This effect may occur by direct cell-to-cell interactions between CD14+ monocyte cells and CD4+ T cells and/or an indirect effect by soluble factor(s) produced by CD14+ monocyte cells. A transwell culture system was used to determine if cell-to-cell direct interactions are required for Foxp3+ cell induction with CD14+ monocyte cells. The culture system comprised tissue culture plates and inserts with corresponding wells. The bottom of each insert well was formed by a nylon membrane with pores smaller than 0.5 micrometer to prevent movement of cells through the membrane, while allowing movement of molecules such as cytokines between the insert and plate wells. With this arrangement, if direct cell-to-cell interaction is required for Foxp3+ cell induction, CD14+ monocyte cells and CD4+ T cells placed in the transwell culture system but separated by a membrane would not lead to induction of Foxp3+ Tregs. On the other hand, if a soluble factor is (or factors) are sufficient to induce Foxp3+ Tregs, separation of CD14+ monocyte cells and CD4+ T cells with the membrane would still lead to induction of Foxp3+ Tregs.

For the investigation, CD4+CD25− T cells from cord blood were placed in the insert wells and stimulated with anti-CD3 and anti-CD28 antibody-coated polystyrene beads. As observed previously (FIG. 2), this form of stimulation does not induce Foxp3+ Tregs. The insert wells were placed in plate wells that either contained or did not contain whole cord blood cells (containing both CD14+ monocyte and CD4+ T cells). After one week of culture, cells were stained with anti-CD127 and Foxp3. As expected, for insert (top) wells placed in plate (bottom) wells that did not contain cord blood cells (upper right panel of FIG. 4), stimulation with the antibody-coated polystyrene beads did not induce Foxp3+ Tregs. In those insert wells placed in plate wells containing total cord blood cells (left panels of FIG. 4), more than 20% of the cells in the insert (top) wells were observed to be Foxp3+ Tregs, evidencing that CD4+ T cells that did not directly interact with CD14+ monocyte cells had become Foxp3+ Tregs. The percentage of Foxp3+ Tregs was comparable between those in the top insert wells (upper left panel of FIG. 4) and bottom plate wells (lower left panel of FIG. 4), suggesting that a soluble factor replaced the ability of CD14+ monocyte cells to induce Foxp3+ Tregs. The level of Foxp3+ Tregs remained the same over a period of three weeks.

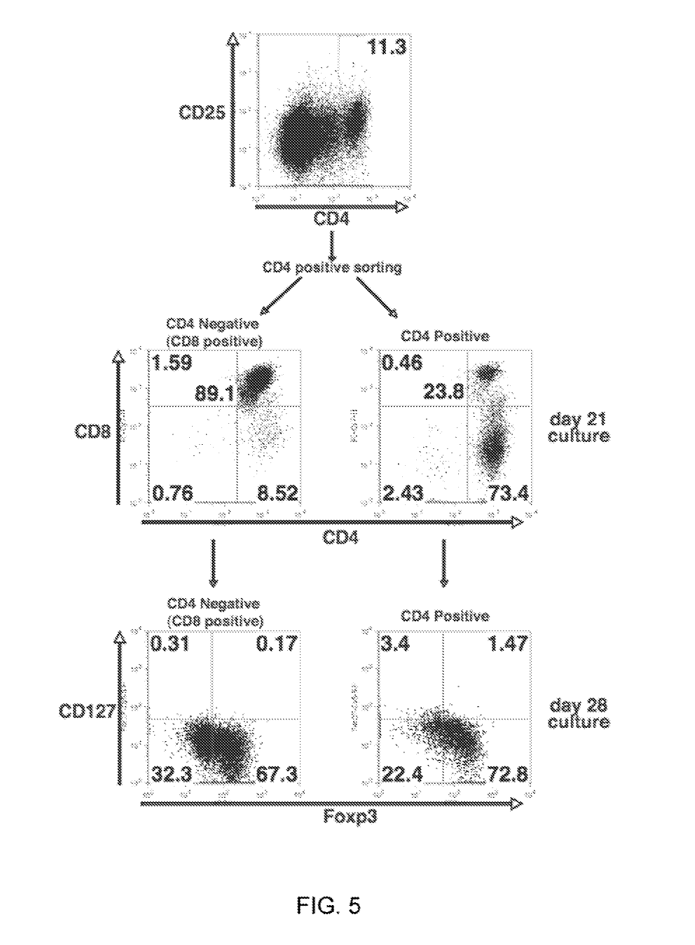

To further investigate the capability of cord blood cells to induce regulatory T cells, T cells for CD4+ and CD4− groups were separated by magnetic cell sorting, as represented in FIG. 5. A majority of CD4− cells were CD8+ (not shown). These cells were mixed with another sample of irradiated T depleted fraction taken from the prior cord blood sample and stimulated with anti-CD3 antibody and IL-2. Three weeks later, the surface antigen phenotypes of cells expanded under the culture conditions were characterized (middle panels of FIG. 5). Cells started from CD4+ population contained about 73% of CD4+CD8− cells and about 23.8% of CD4-CD8+ cells. Cells started from CD4− population contained about 89% of CD8+CD4− cells and about 8.5% of CD4+CD8− cells, indicating that the CD4− population was mainly CD8+ cells. Foxp3 and CD127 (IL-7 receptor) expression by these cells was carried out four weeks after the start of the culture (lower panels of FIG. 5). As observed in the previous culture, the majority (about 72.8%) of the cells expanded from CD4+ T cells were Foxp3+ Tregs. Surprisingly, CD8+ T cells also expressed Foxp3+ at a level comparable to that obtained from CD4+ T cells (about 67.3%).

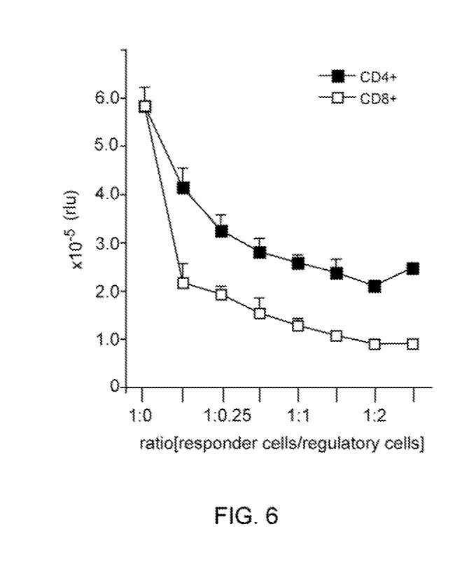

Foxp3 expression by CD8 cells may indicate they have suppression activity similar to CD4+Foxp3+ Tregs. Alternatively, these cells may have different functions than CD4+Foxp3+ Tregs. To determine whether CD8+Foxp3+ T cells are functional Tregs, an in vitro suppression assay was carried out as described above for CD4+ Tregs. Cord blood derived CD8 T cells did not proliferate when they were stimulated in the absence of IL-2, indicating that they are anergic to antigenic stimulation. These cells (regulatory cells) were mixed with the naive syngeneic CD4 T cells (responder cells) from the same cord blood sample in graded doses (1:0 to about 1:4). The responder cells were stimulated with anti-CD3 antibody and irradiated APCs (antigen-presenting cells), and proliferation was measured by the level of ATP (adenosine-5′-triphosphate) in the culture after three days of stimulation using the luciferase-based assay (CellTiter-Glo from the Promega Corporation). As evident from FIG. 6, the CD8+ derived cells showed a potent suppression activity, even stronger than Tregs derived from CD4+ T cells. The data showed that a cord blood environment has the capability of inducing CD8+ Tregs, which have been recently described by several groups (Smith and Kumar, “Revival of CD8+ Treg-mediated Suppression,” Trends Immunol 29, 337-342 (2008)).

To investigate the soluble factor involved in induction of Tregs by CD14+ monocyte cells, testing was conducted to determine whether TGF-β signaling is involved in Foxp3+ Treg induction by cord blood CD14+ monocyte cells. The investigation used SB-431542, which is a known inhibitor for TGF-β receptor kinase. More particularly, SB431542 is a selective inhibitor of TGF-β receptor ALK5 and also blocks ALK4 and ALK7 to a lesser degree. BMP (bone morphogenetic protein) receptors ALK2, 3 and 6 are insensitive to SB431542. Ligands for the receptors that are sensitive to SB431542 include TGF-β1, -β2 and -β3, activins, and nodals.

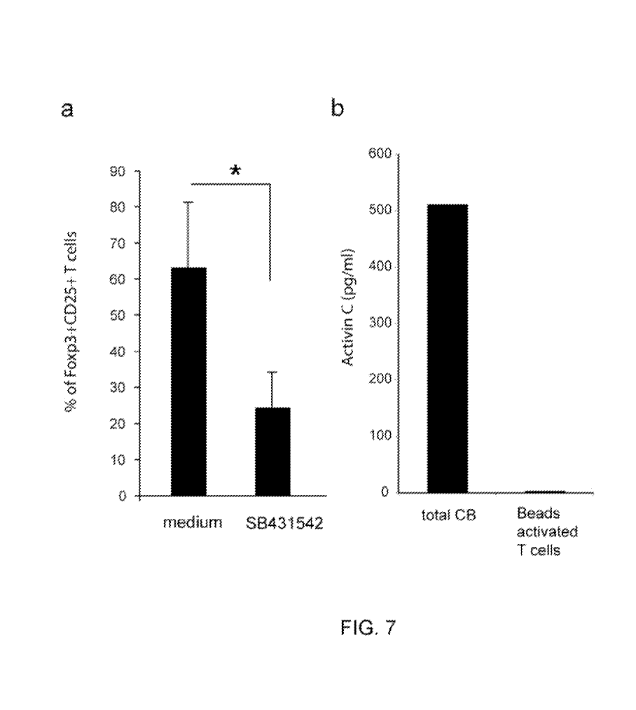

CD4+ T cells from cord blood were treated with anti-CD3 antibody and IL-2 for Treg induction. The graph of FIG. 7a evidences that anti-CD3 stimulation induced more than 60% of CD4+ T cells to be Foxp3+ in three independent samples, and that the addition of SB431542 reduced Foxp3+ cells to about 20% in other samples (the * symbol is used to indicate a significant difference, p<0.005). An effect on cell viability under these conditions was not observed. Thus, the data evidenced that TGF-β was a factor in inducing expression of Foxp3+ by naive T cells, and strongly suggest that TGF-β signaling is required for induction of Tregs by cord blood CD14+ monocyte cells.

To test which TGF-β family is involved, the level of TGF-β family members was examined in a culture supernatant from a total cord blood sample stimulated with anti-CD3 to induce Foxp3+ Tregs. The results are plotted in the graph of FIG. 7b, and show that activin C was present at a high level in the culture supernatant. In contrast, activin C was not present at a significant level in a supernatant of a sample of cord blood T cells stimulated by polystyrene beads coated with anti-CD3 and anti-CD28 (which would not induce Foxp3+, as demonstrated above). These results suggested that activin C may be one of the effector molecules involved in the induction of Foxp3+ Tregs from CD4+ T cells.

While the invention has been described in terms of specific embodiments, it is apparent that other forms could be adopted by one skilled in the art. Accordingly, it should be understood that the invention is not limited to the specific embodiments. It should also be understood that the phraseology and terminology employed above are for the purpose of disclosing the embodiments, and do not necessarily serve as limitations to the scope of the invention. Therefore, the scope of the invention is to be limited only by the following claims.

1. A method of inducing functional regulatory T cells by stimulating T cells with CD14+ monocyte cells obtained from cord blood or adult blood.

2. The method according to claim 1, wherein the T cells are at least one of CD4+ T cells and CD8+ T cells.

3. The method according to claim 2, wherein the T cells are CD4+CD25− T cells.

4. The method according to claim 2, wherein the T cells are CD8+ T cells.

5. The method according to claim 1, wherein the T cells are present in whole lymphocyte/monocyte fraction during stimulation with the CD14+ monocyte cells.

6. The method according to claim 5, wherein the whole lymphocyte/monocyte fraction is obtained from cord blood or adult blood.

7. The method according to claim 1, wherein the T cells are purified from cord blood or adult blood prior to being stimulated with the CD14+ monocyte cells.

8. The method according to claim 1, wherein the CD14+ monocyte cells are cord blood CD14+ monocyte cells.

9. The method according to claim 1, wherein the CD14+ monocyte cells are adult peripheral blood CD14+ monocyte cells.

10. The method according to claim 9, further comprising enriching the CD14+ monocyte cells prior to stimulation of the T cells to induce the functional regulatory T cells.

11. The method according to claim 1, wherein the CD14+ monocyte cells are isolated from cord blood or adult blood prior to stimulation of the T cells to induce the functional regulatory T cells.

12. The method according to claim 1, further comprising treating the CD14+ monocyte cells with at least one stimulator prior to or during the induction of the functional regulatory T cells.

13. The method according to claim 12, wherein the stimulator is at least one chosen from the group consisting of allogenic MHC molecules, antigen peptides (both self and non-self), and antigenic proteins.

14. The method according to claim 12, wherein the stimulator comprises anti-CD3 antibody.

15. The method according to claim 1, wherein the functional regulatory T cells comprise Foxp3+ regulatory T cells.

16. The method according to claim 1, wherein the regulatory T cells are induced by direct contact with the CD14+ monocyte cells.

17. The method according to claim 1, wherein the regulatory T cells are induced by non-direct interaction with the CD14+ monocyte cells.

18. The method according to claim 17, wherein the regulatory T cells are induced by a soluble factor or factors.

19. The method according to claim 1, wherein the functional regulatory T cells are stable for at least three weeks.

20. The method according to claim 1, wherein the induction of the functional regulatory T cells comprises activin C TGF-β signaling.
